(19) United States
(12) Patent Application Publication
(10) Pub. No.: US 2006/0205082 A1
(43) Pub. Date: Sep. 14, 2006

(54) Reaction rate determination

(76) Inventor: John S. Middleton, Fullerton, CA (US)

Correspondence Address: Julio Loza;Sheldon & Mak, 9th Floor, 225 S. Lake Ave., Pasadena, CA, 91101, US

(21) Appl. No.: 11/078,670

(22) Filed: Mar. 10, 2005

(51) Int. Cl. G01N 33/04

(52) U.S. Cl. 436/22

(57) ABSTRACT

One embodiment of the invention provides a method for determining the rate of reaction by including sample points before initiation of the reaction. Absorbance measurements are taken over a plurality of time points, prior to initiation of the reaction, to obtain zero-time absorbance points. The sample is combined with a reagent to obtain a mixture, thereby initiating the chemical reaction. Signal data measurements are then obtained for the mixture. The rate of reaction is then obtained for the mixture. For instance, the rate of reaction is obtained using a linear regression of the zero-time absorbance points and one or more reaction absorbance measurements. The reaction rate obtained may be correlated to known reaction rate data to determine the concentration of a chemical of interest in the sample.

### FIELD

One embodiment of the invention pertains to a method for improving the precision of calculating reaction rates by using test points before and after reaction initiation.

### BACKGROUND

In performing various types of assays, it is often important to determine the reaction rate of a particular chemical reaction. The prior art methods of determining reaction rates lack precision because they do not properly account for blank signal data (data gathered prior to the reaction start) in determining the reaction rate.

U.S. Pat. No. 5,420,042 (Schafer) is directed to establishing the correct concentration of an analyte from an ambiguous reaction rate curve. Schafer proposes running analytical reactions twice, a training run followed by an analysis run, to obtain more accurate data.

U.S. Pat. No. 6,245,569 (Meyers) discloses a method of evaluating the kinetics of coagulation reactions in order to obtain valid data.

### SUMMARY OF THE INVENTION

One embodiment of the invention provides a method for determining the rate of reaction by including sample points before initiation of the reaction. By including initial condition data from several time points prior to the initiation of a reaction, a more accurate measurement of the time “0” condition of the sample or reagent may be made. This results in an “anchoring” of the linear regression curve used to determine the reaction rate, thereby making the resulting analyte concentration data more accurate and precise.

Absorbance measurements are taken over a plurality of time points, prior to initiation of the reaction, to obtain a zero-time absorbance points. The sample is combined with a reagent to obtain a mixture, thereby initiating the chemical reaction. Absorbance data measurements are then obtained for the mixture. The reaction rate is then obtained for the mixture. For instance, the reaction rate is obtained using a linear regression of the zero-time absorbance point(s) and one or more absorbance measurements. The reaction rate obtained may be correlated to known reaction rate data to determine the concentration of a chemical of interest in the sample.

### BRIEF DESCRIPTION OF THE DRAWINGS

FIG. 1 illustrates reaction absorbance measurements for a chemical reaction according to a prior art method.

FIG. 2 illustrates reaction absorbance measurements according to one embodiment of the present invention.

FIG. 3 is a flow diagram illustrating a method for determining a rate of reaction according to one embodiment of the invention.

FIG. 4 is a table illustrating sample reaction measurements for a test performed on a chemistry analyzer according to one embodiment of the invention.

### DETAILED DESCRIPTION

In the following description numerous specific details are set forth in order to provide a thorough understanding of the invention. However, one skilled in the art would recognize that the invention might be practiced without these specific details. In other instances, well known methods, procedures, and/or components have not been described in detail so as not to unnecessarily obscure aspects of the invention.

One embodiment of the invention relates to an improved method for determining reaction rates for chemical assays. In one embodiment, reaction rates are indicative of the concentration of an analyte in the sample. To accurately determine the reaction rate, initial condition data (e.g., blank absorbance data gathered prior to reaction initiation) is aggregated so as to anchor the regression line associated with reaction rate determinations.

Sample measurements are traditionally taken at the initiation of a reaction between a sample and a reagent (i.e., starting at a time “0” of the reaction), or shortly thereafter. The present invention provides a novel improvement to the traditional method of determining reaction rates by measuring the “blank” prior to the initiation of the reaction, preferably over several time points. The “blank” may be either the sample or the reagent, prior to addition of the other component and the subsequent initiation of the reaction. By including the blank data prior to reaction initiation in plotting the reaction curve, a more accurate measurement of the time “0” condition (i.e., the period prior to reaction initiation) of the blank may be made. This measured blank data, can be used to make the resulting reaction rate determination more accurate and improve assay precision.

When testing for analyte concentration in a sample, sample measurements typically begin at the initiation of a reaction between the sample and a detection reagent (i.e., starting at time “0” of the reaction), or shortly thereafter. Measurements may be taken prior to the start of the reaction to establish a baseline condition of the starting sample or of the reagent (i.e., the “blank” or initial condition). One embodiment of the invention improves such testing methods by measuring the “blank” over several time points prior to the initiation of a reaction. By including initial condition data from several time points prior to the initiation of a reaction, a more accurate measurement of the time “0” condition of the sample or reagent may be made. This results in an “anchoring” of the linear regression curve used to determine the reaction rate, thereby making the resulting analyte concentration data more accurate and improving assay precision.

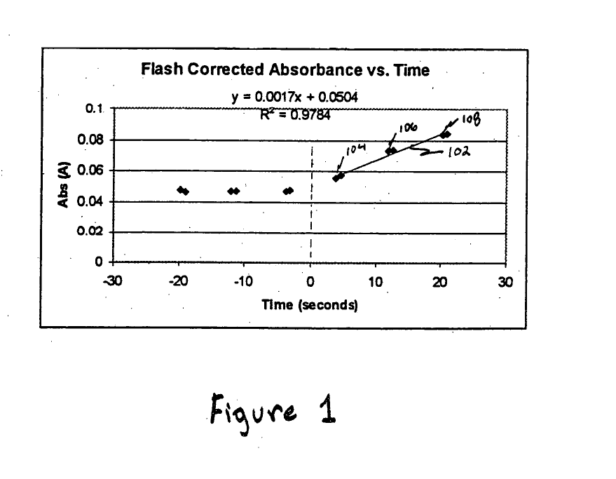

FIG. 1 illustrates reaction absorbance measurements for a chemical reaction according to a prior art method. As illustrated, absorbance measurements 104, 106, and 108 are used to determine a rate of reaction for a given sample and a corresponding reagent. Linear regression 102 is applied according to a prior art method to ascertain a rate of reaction. However, such prior art methods do not consider the “blank” measurements prior to, or at, time “0”. As a result, this linear regression rate of reaction determination does not use measurements at time “0” and has less data points. Consequently, determining the rate of reaction using linear regression is imprecise.

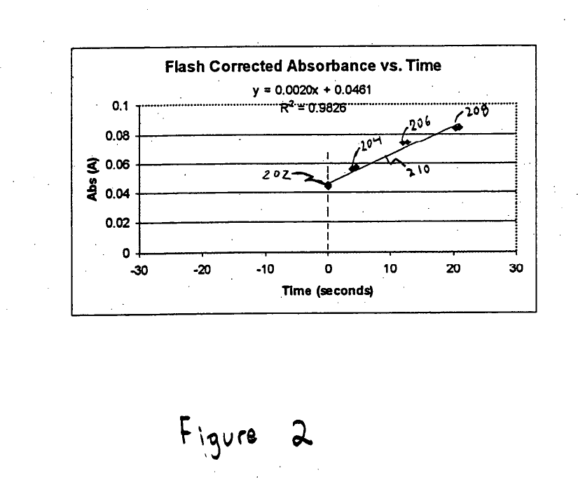

FIG. 2 illustrates reaction absorbance measurements according to one embodiment of the present invention. This linear regression reaction rate calculation includes aggregate points 202 of one or more “blank” measurements prior to time “0”. Aggregate points 202 are placed at time “0” and used as part of linear regression 210 with other reaction absorbance points 204, 206, and 208. By using the time “0” measurements, the system is able to more accurately determine a reaction rate via linear regression 210.

With the present invention, blank absorbance data 202 is included in the rate determination linear regression 210. The blank data 202 corresponds to measurements at time less than zero (time<0) seconds, before the sample is combined with a reagent. The time values for the blank absorbance data 202 are set to zero as the blank data represents the state of the reaction at time zero. For instance, several absorbance measurements may be taken prior to time zero to obtain one or more blank data points 202 used at time zero (time=0).

According to one embodiment of the invention, blank absorbance is corrected for sample dilution. For example, if the sample volume is ten (10) micro-liters and the reagent volume is two hundred thirty (230) micro-liters, the Corrected Blank Absorbance is (230/240)×Absorbance.

It is important to note that, in order for this method to be applied, the blank absorbance must be temporarily stable such that, taking an average of that absorbance and using that average (corrected for dilution as described above), accurately represents the signal at the beginning of the reaction.

The inclusion of blank data improves the robustness of the linear regression rate determination process by heavily anchoring the line at the time zero or at a time immediately before reaction initiation. Thus, the present invention provides improved assay precision performance.

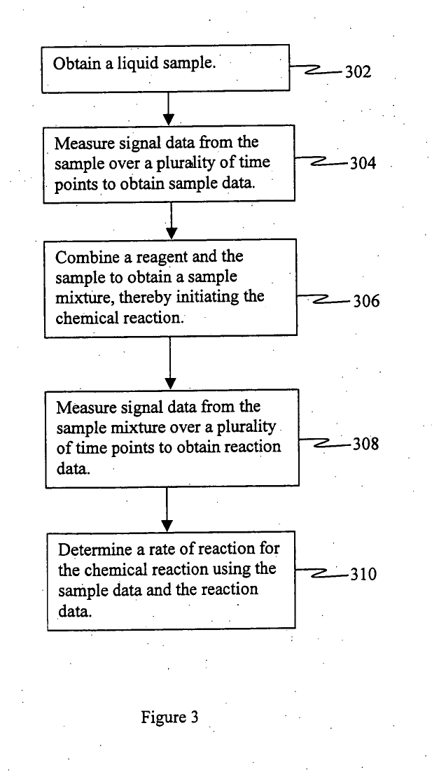

FIG. 3 is a flow diagram illustrating a method for determining a rate of reaction according to one embodiment of the invention. A sample of the liquid sample, reagent, analyte, etc., is obtained 302. One or more absorbance measurements are taken over a plurality of time points to obtain zero-time absorbance points. The sample is combined with a reagent to obtain a mixture, thereby initiating the chemical reaction 306. Absorbance data measurements are then obtained for the mixture 308. The rate of reaction is then obtained for the mixture 310. For instance, the rate of reaction is obtained using a linear regression of the zero-time absorbance points and one or more reaction absorbance measurements. In one embodiment of the invention, the reaction rate obtained is correlated to known reaction rate data to determine the concentration of the analyte of interest in the sample.

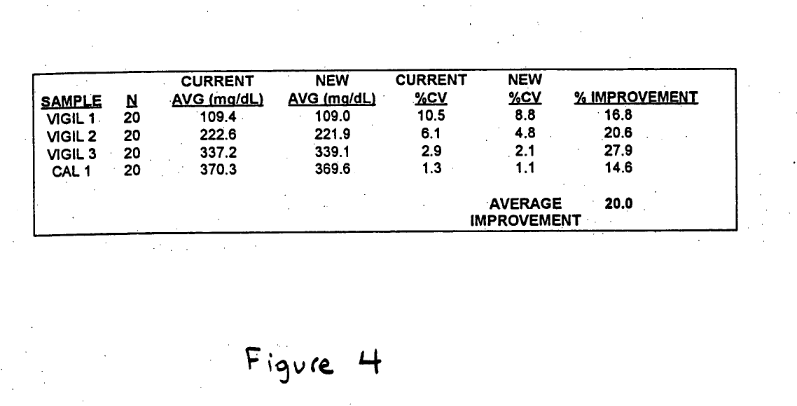

FIG. 4 is a table illustrating sample reaction measurements for a test performed on a chemistry analyzer, such as the Synchron LX® by Beckman Coulter, according to one embodiment of the invention. However, the method of the present invention may be performed on any analytical assay that utilizes rate determinations at the beginning of the analytical reaction. The table in FIG. 4 summarizes a comparison between the prior art method (Current) and the method of the present invention (New) for calculating analyte concentration. The assay in this test is triggered LX IgA. As illustrated in FIG. 4, four different test samples (Vigil 1, Vigil 2, Vigil 3, and Cal 1) are assayed twenty times each. The average and coefficient of variation (i.e., relative standard deviation) is determined for each sample and for each method (current and new). The percentage of improvement of the new method over the prior art method is approximately twenty percent on average.

Note that not all assays can be improved using the method of the present invention. In particular, the nature of the chemistry must be such that the corrected blank absorbance value is stable and representative of the beginning of the reaction.

While certain exemplary embodiments have been described and shown in the accompanying drawings, it is to be understood that such embodiments are merely illustrative of and not restrictive on the broad invention, and that this invention not be limited to the specific constructions and arrangements shown and described, since various other modifications are possible. Those skilled, in the art will appreciate that various adaptations and modifications of the just described preferred embodiment can be configured without departing from the scope and spirit of the invention. Therefore, it is to be understood that, within the scope of the appended claims, the invention may be practiced other than as specifically described herein.

What is claimed is:

1. A method for determining the rate of a chemical reaction, comprising the following steps:
(a) measuring signal data from either a sample or a reagent over a plurality of time points to obtain blank data;
(b) combining the reagent and the sample after step (a) to obtain a mixture, thereby initiating the chemical reaction;
(c) measuring signal data from the mixture over a plurality of time points to obtain reaction data; and
(d) determining a rate of reaction for the chemical reaction using the blank data and the reaction data.

2. The method of claim 1, wherein the steps of measuring signal data from either the sample or the reagent and measuring signal data from the mixture include measuring optical signal data.

3. The method of claim 1, wherein the steps of measuring signal data from either the sample or the reagent and measuring signal data from the mixture include measuring voltage data.

4. The method of claim 1, wherein the steps of measuring signal data from either the sample or the reagent and measuring signal data from the mixture include measuring conductivity data.

5. The method of claim 1, wherein the steps of measuring signal data from either the sample or the reagent and measuring signal data from the mixture include measuring temperature data.

6. The method of claim 1 further comprising:
correlating the reaction rate to known reaction rate data to determine a concentration of a chemical of interest in the sample.

7. The method of claim 1 wherein the blank data includes blank absorbance data, further comprising:
determining a corrected absorbance data for either the sample or the reagent that compensates for sample dilution.

8. The method of claim 1 further comprising:
obtaining the sample, wherein the sample is a liquid sample.

9. The method of claim 1 further comprising:
obtaining the reagent, wherein the reagent is a liquid reagent.

10. The method of claim 1 further comprising:
applying linear regression to the blank data and the reaction data to determine the rate of reaction for the chemical reaction.

11. A method of determining the concentration of an analyte of interest in a sample, comprising the following steps:
(a) measuring signal data from either a sample or a reagent over a plurality of time points to obtain blank data;
(b) combining the reagent and the sample to form a mixture after step (a), thereby initiating a chemical reaction if the sample contains the analyte of interest;
(c) measuring signal data from the mixture over a plurality of time points to obtain reaction data;
(d) determining a rate of reaction for the chemical reaction using the blank data and the reaction data; and
(e) correlating the reaction rate to known reaction rate data to determine the concentration of the analyte of interest in the sample.

12. The method of claim 11 further comprising:
obtaining the sample to be tested for the analyte of interest, wherein the sample is a liquid sample; and
obtaining the reagent, wherein the reagent is a liquid reagent.

13. The method of claim 11, wherein the steps of measuring signal data from either the sample or the reagent and measuring signal data from the mixture include at least one step of
measuring optical signal data;
measuring voltage data;
measuring conductivity data; or
measuring temperature data.

14. The method of claim 11, further comprising:
determining a corrected blank signal data for either the sample or the reagent that compensates for sample dilution.

15. The method of claim 11 further comprising:
applying linear regression to the blank data and the reaction data to determine the rate of reaction for the chemical reaction.

16. The method of claim 11 wherein the linear regression is weighted by the plurality blank data points.

17. A machine-readable medium having one or more instructions for determining the concentration of a chemical of interest in a sample, which when executed by a processor, causes the processor to perform operations comprising:
(a) obtaining a liquid sample;
(b) measuring signal data from either a sample or a reagent over a plurality of time points to obtain blank data;
(c) combining the reagent and the sample to form a mixture after step (b), thereby initiating a chemical reaction;
(d) measuring signal data from the mixture over a plurality of time points to obtain reaction data;
(d) applying linear regression to the blank data and the measured reaction data to determine a rate of reaction for the chemical reaction; and
(e) correlating the reaction rate to known reaction rate data to determine the concentration of the first chemical of interest in the sample.
